(19) United States
(12) Patent Application Publication
(10) Pub. No.: US 2016/0374815 A1
(43) Pub. Date: Dec. 29, 2016

(54) PROSTHESIS FOR ANATOMICAL OR REVERSE RECONSTRUCTION OF THE SHOULDER JOINT

(71) Applicant: MEDACTA INTERNATIONAL SA, Catel San Pietro (CH)

(72) Inventors: Alberto SICCARDI, SONVICO (CH); Francesco SICCARDI, VICO MORCOTE (CH); Massimiliano BERNARDONI, FIGINO (CH); Matteo ZANIRATO, ADRIA (IT)

(21) Appl. No.: 14/901,804

(22) PCT Filed: Jul. 4, 2014
(86) PCT No.: PCT/IB2014/062862

§ 371 Date: Dec. 29, 2015

(30) Foreign Application Priority Data
Jul. 4, 2013 (IT) MI2013A001127

(51) Int. Cl. A61F 2/40

(52) U.S. Cl. CPC A61F 2/4003; A61F 2/4081; A61F 2002/30649

(57) ABSTRACT

A prosthesis (10) for the reconstruction of the shoulder joint comprises a glenoid prosthesis (11) comprising an attachment support for the glenoid (13) and a joint element of the glenoid (14) and a humeral prosthesis (12) comprising an attachment support for the humerus (17) and a joint element of the humerus (15). The joint elements of the glenoid (14) and the humerus (15) are shaped in such a way as to achieve a spherical-type coupling therebetween for the artificial reconstruction of the shoulder joint. The joint elements of the glenoid (14) and the humerus (15) are constrained in a removable way by said attachment supports for the glenoid (13) and the humerus (17). The joint element of the humerus (15) is made of plastic material, in particular of polyethylene, and the joint element of the glenoid (14) is made of metallic material, in particular of a cobalt-chromium alloy.

The present invention relates to a prosthesis for the reconstruction of the shoulder joint.

In particular, the present invention relates to a prosthesis capable to allow both anatomical and “reverse” reconstruction of the shoulder joint.

In general, the shoulder joint prostheses today known comprise an element associated with the glenoid and an element associated with the humerus, each of such elements comprising a portion which attaches to the bone and a joint front portion.

The joint front portions are the one hemisphere-shaped and the other one shaped with a complementary convexity.

In the case of anatomical prosthesis, the front portion of the humeral joint is hemispherical.

In case of reverse prosthesis, instead, the front portion of the glenoid joint is hemispherical.

There are different types of prostheses for the reconstruction of the shoulder joint currently on the market which, however, have some drawbacks, related in particular to:

detachment from the glenoid;

failure or breakage of the front portion of the glenoid joint; and

difficult transition from the anatomical configuration to the reverse configuration.

The reasons for such drawbacks will be made clear below.

The detachment of the glenoid prosthesis is due to the fact that it is currently widespread the practice of using gluing elements as means for fixing the prosthesis to the glenoid.

This gluing, known in the field as “cementation technique”, loses its efficacy as time passes, thus not assuring the correct positioning of the prosthesis.

Breakage of the front portion of the glenoid joint, made of polyethylene, is due to the fact that, because of the shape of the glenoid itself, it is not possible to increase the thickness of this portion. This reduced thickness, together with the choice of polyethylene as material, results in a high failure rate.

Finally, there is a present difficulty in transitioning from the anatomical configuration to the reverse configuration, precisely because of the constraint in gluing the prosthesis to the glenoid.

In fact, it would be necessary to strain the glenoid, which is fragile per se, by removing the current prosthesis to replace it with a reverse one.

In view of this prior art, the object of the present invention is to provide prostheses for the reconstruction of the shoulder joint that is alternative to those known, extremely efficient and capable of achieving the following results:

reducing the risk of detachment from the glenoid;

eliminating the failure or breakage of the front portion of the glenoid joint; and

making the transition from the anatomical configuration to the reverse configuration simple.

In general, the present invention achieves the results described above by reversing, in a way that is absolutely innovative and which contradicts the known standard, the tribological characteristics of the coupling at the base of the prosthesis for the reconstruction of the shoulder joint.

In particular, it is expected that the front portion of the glenoid joint will be made of a metallic material, and the corresponding one of the humerus of a plastic material.

The portions for attachment to the glenoid and to the humerus are made of the “cementless” type of metallic material, i.e. fixed in place by means of threaded elements, and are configured to removably receive the joint portions thereof. In this way, it will be much easier to switch from an anatomical configuration to a reverse configuration.

Further characteristics of the invention will be pointed out by the dependent claims and by the embodiments shown in the figures.

The characteristics and the advantages of prostheses for the reconstruction of shoulder joints according to the present invention will become more evident from the following, exemplary and non-limiting, description, referring to the attached schematic drawings, in which:

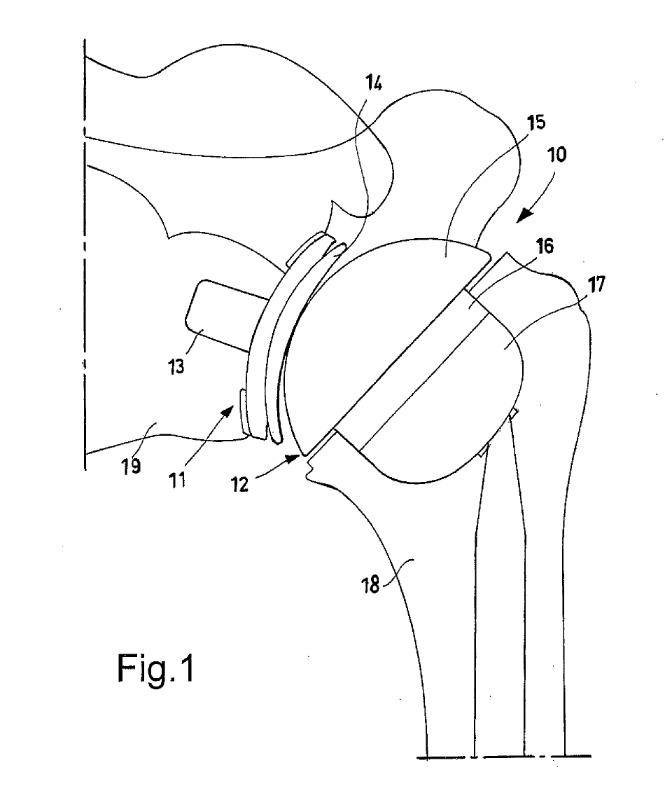

FIG. 1 shows an exemplary embodiment of a prosthesis for the reconstruction of a shoulder joint according to the present invention in use in anatomical configuration;

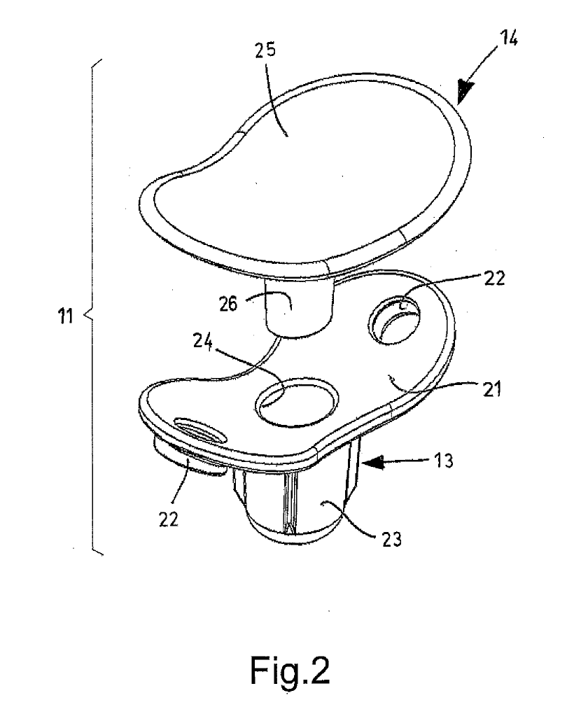

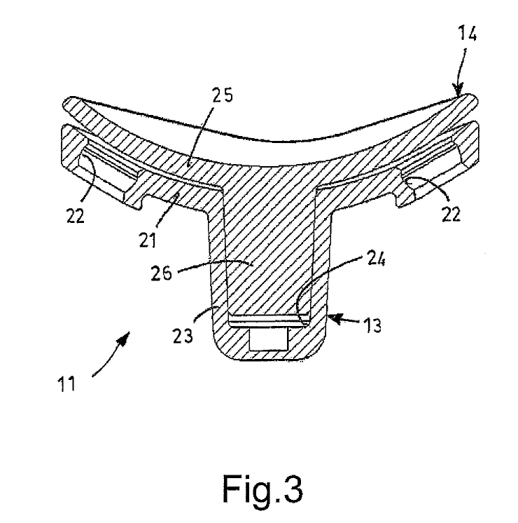

FIGS. 2 and 3 show exploded and sectional views of the portion of the prosthesis in FIG. 1 relative to the glenoid;

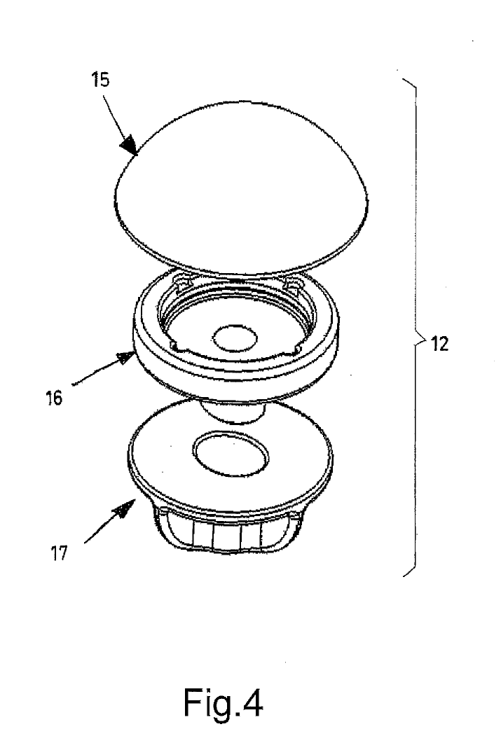

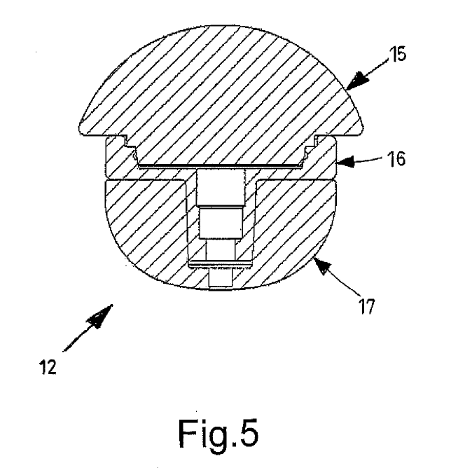

FIGS. 4 and 5 show exploded and sectional views of the portion of the prosthesis in FIG. 1 relative to the humerus; and

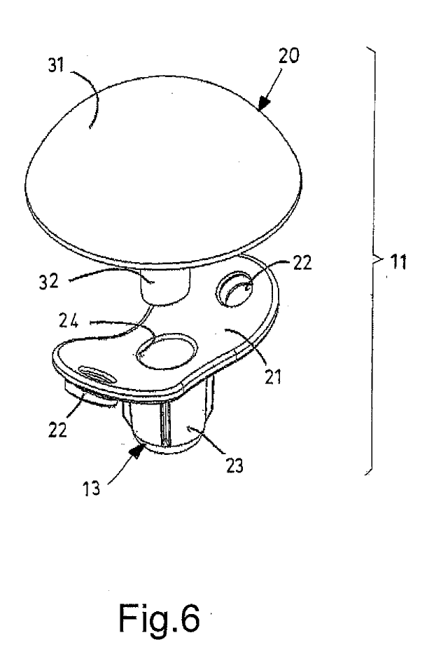

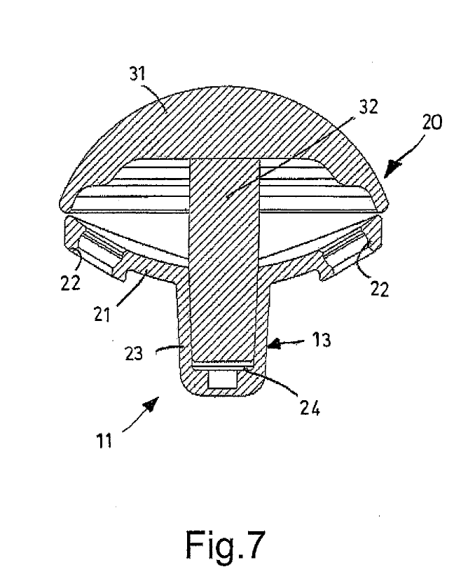

FIGS. 6 and 7 show exploded and sectional views of the portion relative to the glenoid of the prosthesis of the present invention in reverse configuration.

Referring to the figures, 10 denotes a prosthesis for the reconstruction of the shoulder joint according to the present invention.

This prosthesis 10 comprises:

a glenoid prosthesis 11 comprising an attachment support for the glenoid 13 and a joint element for the glenoid 14; and

a humeral prosthesis 12 comprising an attachment support for the humerus 17 and a joint element for the humerus 15.

In a way per se known, the joint elements of the glenoid 14 and the humerus 15 are shaped in a way such as to achieve a spherical coupling therebetween for the artificial reconstruction of the natural shoulder joint.

Advantageously, the joint elements of both the glenoid 14 and the humerus 15 are removably constrained by their respective attachment supports 13 and 17.

Particularly, according to the invention, the joint element of the humerus 15 is made of a plastic material while the joint element of the glenoid 14 is made of a metallic material.

Such a tribological coupling, besides the profile of the joint elements that rely on the use of the prosthesis in an anatomical or reverse configuration, is completely innovative and offers a sort of overcoming of a technical prejudice, in that the joint element of the humerus 15 should be made of metal and the joint element of the glenoid 14 should be made of plastic. As shown, the joint element of the glenoid 14 is of the type which can be constrained to the glenoid by means of threaded elements and it is made of a cobalt-chromium alloy.

Preferably, the joint element of the humerus 15 is made of polyethylene, while the attachment supports to both the glenoid 13 and the humerus 17 are made of a metallic material, for example a titanium alloy.

In the shown embodiment, the attachment support to the glenoid 13 is of a T-shaped type with a convex transverse portion 21 provided at the ends of holes 22 for the passage of threaded elements.

The stem portion 23 is provided with a blind hole 24 for receipt of the joint element of the glenoid 14.

The joint element of the glenoid 14 may be a concave surface 25 internally provided with a stem 26 for coupling with the blind hole 24 of the glenoid attachment support 13, or a hemispherical body 31 also provided with a stem 32, depending on whether the prosthesis is used in an anatomical or reverse way.

The portion of humeral prosthesis 12 comprises, in addition, an intermediate support 16 between the attachment support for the humerus 17 and the joint element of the humerus 15, where the attachment support for the humerus 17 is made of a titanium alloy and the intermediate support 16 is made of a cobalt-chromium alloy.

In case of anatomical prosthesis, the intermediate support 16 is associated with a hemispherical-shaped, humeral joint element 15.

In case of reverse prosthesis, the intermediate support 16 is associated with a convex-shaped, humeral joint element 15.

However, in both cases, the humeral joint element 15 is made of polyethylene.

It has thus been seen that a prosthesis for the reconstruction of the shoulder joint according to the present invention achieves the previously outlined objectives.

In fact, the novel tribological coupling between the joint portion of the glenoid made of cobalt-chromium and the one made of polyethylene of the humerus allows to achieve the following results:

reducing the risks of detachment from the glenoid;

suppressing failure or breakage of the front portion of the glenoid joint; and

making simple the transition from the anatomical configuration to the reverse configuration.

The reduction of the risks of detachment from the glenoid is due to mechanical constraint while breakage of the front portion of the glenoid joint is avoided thanks to the innovative choice of using the cobalt-chromium alloy.

This alloy, in fact, even with reduced thickness, offers high resistance to the front portion of the glenoid joint.

The ease of transition from the anatomical configuration to the reverse configuration is given by the configuration of the attachment portions which are “cementless” and which provide a releasable snap-coupling with the joint portions.

The prosthesis for shoulder joint reconstruction of the present invention thus conceived is subject to numerous modifications and variations, all within the same inventive concept; in addition, all the details can be replaced by technically equivalent elements. In practice, the materials used, as well as their dimensions, can be of any type depending on the technical requirements.

1. A prosthesis for the reconstruction of the shoulder joint, comprising:
a glenoid prosthesis comprising an attachment support for the glenoid and a joint element of the glenoid; and
a humeral prosthesis comprising an attachment support for the humerus and a joint element of the humerus;
said joint elements of the glenoid and the humerus being shaped in such a way as to achieve a spherical-type coupling therebetween for the artificial reconstruction of the shoulder joint;
said joint elements of the glenoid and the humerus being constrained in a removable way by said attachment supports for the glenoid and the humerus; said joint element of the humerus being made of plastic material and said joint element of the glenoid being made of metallic material;
said prosthesis being characterized in that said joint element of the glenoid is made of a cobalt-chromium alloy, and the joint element of the humerus is made of polyethylene.

2. The prosthesis according to claim I characterized in that said joint element of the glenoid is of the type that can be constrained to the glenoid by means of threaded elements.

3. The prosthesis according to claim 1, in that said attachment supports for the glenoid and the humerus are made of a metallic material.

4. The prosthesis according to claim 1, characterized in that said attachment supports for the glenoid and the humerus are made of a titanium alloy.

5. The prosthesis according to claim 1, characterized in that said attachment support for the glenoid is of the T-shaped type with a convex transverse portion provided at the ends of holes for the passage of threaded elements, the stem portion being provided with a blind hole for receipt of said joint element of the glenoid.

6. The prosthesis according to claim 5, characterized in that said joint element of the glenoid comprises a concave surface internally provided with a stem for coupling with said blind hole of said attachment support for the glenoid.

7. The prosthesis according to claim 5, characterized in that said joint element of the glenoid comprises a semi-spherical body internally provided with a stem for coupling with said blind hole of said attachment support for the glenoid.

8. The prosthesis according to claim 5, characterized in that said humeral prosthesis comprises an intermediate support between said attachment support for the humerus and said joint element of the humerus.

9. The prosthesis according to claim 5, characterized in that said attachment support for the humerus is made of titanium and said intermediate support is made of a cobalt-chromium alloy.
